(19) United States
(12) Patent Application Publication
(10) Pub. No.: US 2024/0278028 A1
(43) Pub. Date: Aug. 22, 2024

(54) DEVICE FOR LEFT ATRIAL APPENDAGE

(71) Applicant: BOSTON SCIENTIFIC SCIMED, INC., Maple Grove, MN (US)

(72) Inventors: Jerry Wang, Edina, MN (US); Joshua Mark Inouye, Brooklyn Park, MN (US); James M. Anderson, Corcoran, MN (US)

(73) Assignee: BOSTON SCIENTIFIC SCIMED, INC., MAPLE GROVE, MN (US)

(21) Appl. No.: 18/444,917

(22) Filed: Feb. 19, 2024

Related U.S. Application Data
(60) Provisional application No. 63/446,925, filed on Feb. 20, 2023.

(51) Int. Cl. A61N 1/39; A61B 17/00

(52) U.S. Cl. CPC A61N 1/395; A61B 17/00234; A61B 2017/0011; A61B 2017/00243

(57) ABSTRACT

Blood stagnation within a left atrial appendage (LAA) may be reduced by creating movement within the LAA. A variety of different actuatable devices, including magnetically actuated devices, may be implanted within the LAA. The actuatable device may be caused to become actuated, thereby causing the motion within the LAA. In some cases, causing the actuatable device to become actuated may include application of a magnetic field. In some cases, causing the actuatable device to become actuated may include subjecting the actuatable device to blood flow within the LAA.

### CROSS REFERENCE TO RELATED APPLICATIONS

The present application claims the benefit of and priority to U.S. Provisional Patent Application No. 63/446,925, filed Feb. 20, 2023, the disclosure of which is incorporated herein by reference.

### TECHNICAL FIELD

The disclosure relates generally to medical devices and more particularly to medical devices that are adapted for preventing blood stagnation within a left atrial appendage.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a method for reducing blood stagnation within a left atrial appendage (LAA) using an actuatable device. The method includes disposing an actuatable device within the LAA that is adapted to create blood flow within the LAA, and actuating the actuatable device in order to reduce blood stagnation within the LAA.

Alternatively or additionally, actuating the actuatable device may include subjecting the actuatable device to blood flow within the LAA.

Alternatively or additionally, the actuatable device may include an oscillating piston pump.

Alternatively or additionally, the actuatable device may include a propeller.

Alternatively or additionally, the actuatable device may further include a motor adapted to rotate the propeller.

Alternatively or additionally, the propeller may be adapted to be actuatable via motion within the LAA.

Alternatively or additionally, the actuatable device may include a piezoelectric material and a battery.

Alternatively or additionally, normal motion during times without atrial fibrillation may be used to charge the battery, and energy may be released via the piezoelectric material during times of atrial fibrillation.

Alternatively or additionally, the actuatable device may include a housing adapted to be secured within the LAA, a housing inlet that is adapted to allow blood to flow into the actuatable device, a housing outlet that is adapted to allow blood to flow out of the actuatable device, a power generation element adapted to harness the blood flow to generate power, and a battery that is charged by the power generated by the power generation element.

Alternatively or additionally, the actuatable device may be adapted to recognize periods of atrial fibrillation when the power generated by the power generation element drops below a threshold.

Another example may be found in a method for reducing blood stagnation within a left atrial appendage (LAA) using an actuatable device. The method includes disposing an actuatable device within the LAA that is adapted to create blood flow within the LAA, monitoring for indications of atrial fibrillation, and in response to detecting atrial fibrillation, actuating the actuatable device in order to reduce blood stagnation within the LAA.

Alternatively or additionally, the method may further include ceasing operation of the actuatable device when a normal heart rhythm is once again detected.

Alternatively or additionally, atrial fibrillation may be detected by recognizing a reduction in generated power.

Another example may be found in an actuatable device for reducing blood stagnation within a left atrial appendage (LAA). The actuatable device includes structure adapted to anchor the actuatable device within the LAA and a moveable element that is constrained by the structure but is movable relative to the structure in response to an applied stimuli. Movement of the moveable element in response to the applied stimuli causes blood flow within the LAA, thereby reducing blood stagnation.

Alternatively or additionally, the applied stimuli may include movement of cardiac tissue during a cardiac cycle.

Alternatively or additionally, the applied stimuli may include an applied current.

Alternatively or additionally, the moveable element may include a propeller.

Alternatively or additionally, the actuatable device may further include a motor adapted to rotate the propeller.

Alternatively or additionally, the applied current may be externally provided.

Alternatively or additionally, the applied current may be internally generated.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional view of an LAA (left atrial appendage);

FIG. 2A is a schematic view of an illustrative actuatable device for reducing stagnation within the LAA;

FIG. 2B is a schematic view of an illustrative actuatable device for reducing stagnation within the LAA;

FIG. 3 is a schematic view of an illustrative actuatable device including a propeller and a bi-stable element;

FIGS. 4 through 6 illustrate a process of delivering the illustrative actuatable device of FIG. 3;

FIG. 7 is a schematic view of an illustrative actuatable device including an oscillating piston pump;

FIG. 8 is an end view of the illustrative actuatable device of FIG. 7;

FIG. 9 is a schematic view of an illustrative actuatable device including a propeller;

FIG. 10 is a schematic view of an illustrative actuatable device including a propeller;

FIG. 11 is a schematic view of an illustrative actuatable device including a propeller;

FIG. 12 is a schematic view of an illustrative actuatable device;

FIG. 13 is a schematic view of the illustrative actuatable device of FIG. 16, shown in action;

FIG. 14 is a schematic view of an illustrative actuatable device;

FIG. 15 is a schematic view of an illustrative actuatable device; and

FIGS. 16 through 18 are schematic views of an illustrative actuatable device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following figures illustrate selected components and/or arrangements of an implant for occluding the left atrial appendage, a system for occluding the left atrial appendage, and/or methods of using the implant and/or the system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

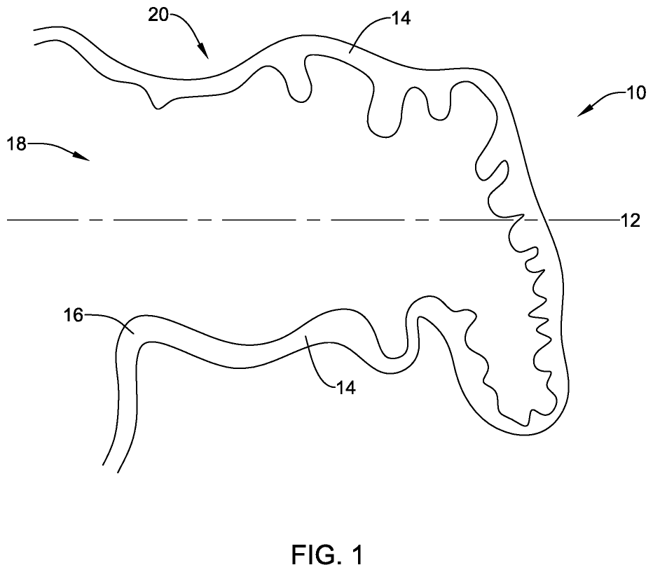

FIG. 1 is a partial cross-sectional view of a left atrial appendage 10. In some embodiments, the left atrial appendage (LAA) 10 may have a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12.

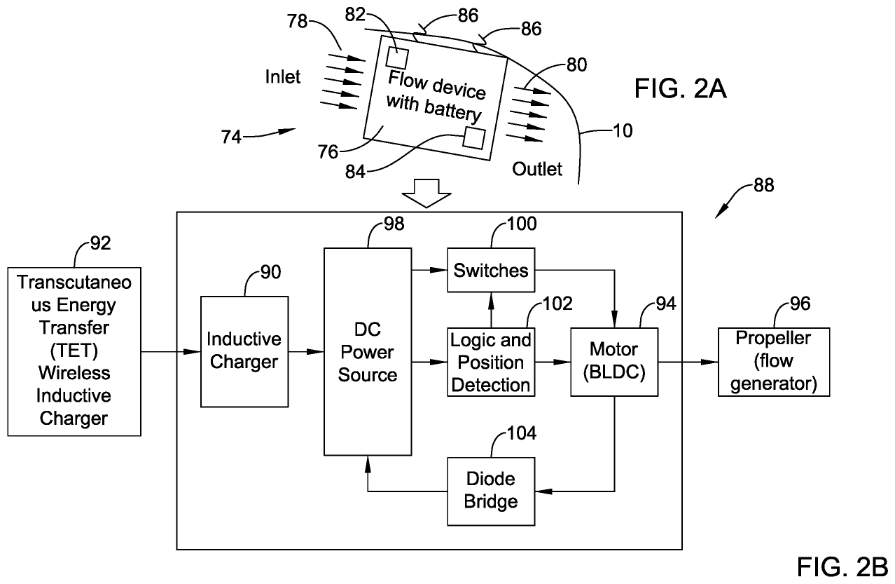

In some cases, and particularly during times of atrial fibrillation, blood flow within the LAA 10 may become stagnant. Stagnant blood flow within the LAA 10 may lead to blood clots, which can lead to stroke. In some cases, creating blood flow within the LAA 10 may help to reduce stagnation and thus reduce the likelihood of blood clot formation, especially during atrial fibrillation. FIGS. 2 through 18 provide examples of actuatable devices that may be implanted within the LAA 10 in order to reduce stagnation. In some cases, these actuatable devices may operate at all times. In some cases, these actuatable devices may only operate when atrial fibrillation has been detected. In some cases, these actuatable devices may be at least partially driven by blood moving within the heart. In some cases, these actuatable devices may be at least partially driven by movement of the heart itself, as the heart beats. It will be appreciated that the heart twists and squeezes with each heartbeat.

FIG. 2A is a schematic view of an actuatable device 74 deployed within the LAA 10. The actuatable device 74 a housing 76 that may be adapted to be implanted in any of a number of different locations within the LAA 10. In some cases, the housing 76 may be adapted to be implanted along the lateral wall 14 of the LAA 10. In some cases, the housing 76 may be adapted to be implanted at other locations within the LAA 10, for example. The housing 76 includes an inlet 78 that allows blood to flow into the housing 76 and an outlet 80 that allows blood to flow out of the housing 76.

During normal heart rhythms, blood may be caused to flow into the inlet 78 and out of the outlet 80. This blood flow can engage a power generation element 82 such as a propeller that is adapted to harness the blood flow to generate power. This may be considered as an example of an internally generated electric current. The power generation element 82 may also include an electric motor that is adapted to drive the propeller into rotation in order to generate blood flow when blood flow is otherwise not causing the propeller to rotate. A battery 84, which may be a rechargeable battery, for example, may be charged by the power generated by the power generation element 82. During normal heart rhythm, the blood flowing through the housing 76 is used to charge the battery 84.

When atrial fibrillation is detected, the energy stored in the battery 84 may be used to actively power the power generation element 82 to create blood flow. Because atrial fibrillation disrupts normal blood flow within the heart, particularly within the atria, a drop in power generation may be an indication that atrial fibrillation has begun. In some cases, the power generation may drop below a threshold that is used as a suggestion that atrial fibrillation is occurring. The housing 76 may be secured relative to the LAA 10 via anchors 86. The anchors 86 may take any of a variety of forms, and may mechanically secure the housing 76 in place relative to the LAA 10.

FIG. 2B is a schematic view of an actuatable device 88 that includes an inductive charger 90 that may be charged using a wireless inductive charger 92. The power transferred to the inductive charger 90 is used to power a motor 94 that drives a propeller 96. The motor 94 is a BLDC motor using DC power. The DC power source 98 provides DC power to the motor 94. The wireless inductive charger 92 and the inductive charger 90 are AC. There is an AC to DC conversion between the inductive charger 90 and the DC power source 98. Power may be inductively provided to the inductive charger 90 using an external power unit (not shown) and thus may be considered as being an externally provided electric current. In some cases, an external power unit may be used to periodically provide power that is used to recharge the DC power source 98, in order to keep the DC power source 98 charged and ready for use if needed. In some cases, an external power unit may be used to provide power only when atrial fibrillation has been detected. The external power unit may be held to the patient's chest in order to inductively provide power. In some cases, the external power unit may be part of a vest that the patient wears when needed, for example.

A charging circuitry includes the DC power source 98, switches 100, logic and position detection 102 and a diode bridge 104. In some cases, the diode bridge 104 provides voltage rectification. The logic and position detector 102 provides pole position feedback for commutation control. The switches 100 provide an electromagnetic conversion by switching stator flux. The motor 94 may be a 2-pole magnet rotor and BLDC stator (brushless DC motor).

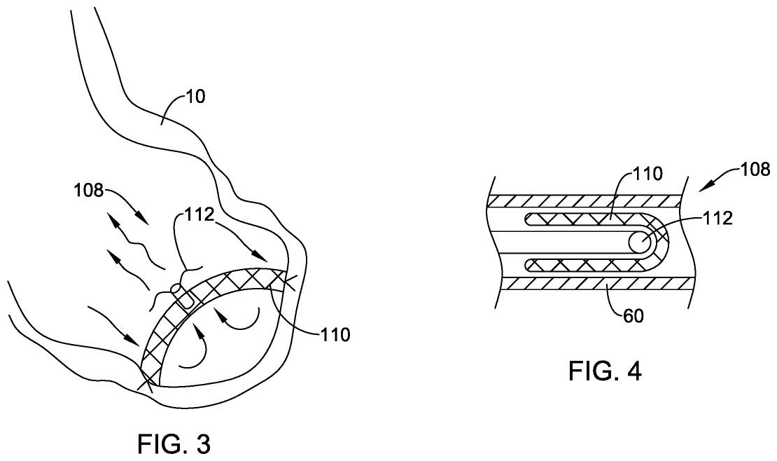

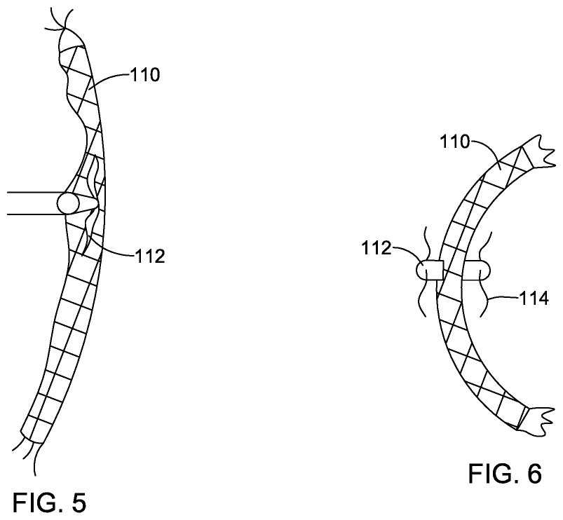

FIG. 3 is a schematic view of an illustrative actuatable device 108 deployed within the LAA 10. The actuatable device 108 includes a bi-stable apparatus 110 and a propeller 112 that is secured relative to the bi-stable apparatus 110. In some cases, physical movement of the heart may cause the bi-stable apparatus 110 to flex between a first position and a second position, or between a concave configuration and a convex configuration. This flexibility is also useful for delivery of the actuatable device 108, as will be discussed with respect to FIGS. 4 and 5. The bi-stable apparatus 110 may be formed of any suitable material.

Rotation of the propeller 112, which may be caused in part by movement of the bi-stable apparatus 110, may generate blood flow within the LAA 10 during times of atrial fibrillation. In some instances, generating sufficient blood flow to create eddies within the LAA 10 is enough to prevent or at least reduce stagnation within the LAA 10. By reducing stagnation, the risk of clot development may be reduced. In some cases, an electric motor driven by inductively provided electrical power may be used to actuate the propeller 112. For example, an external inductive charger could be used to inductively provide power to a charging circuit coupled with the electric motor and propeller 112.

FIG. 4 is a schematic view of the actuatable device 108 disposed within a delivery device 60. In some cases, the delivery device 60 may be a delivery catheter, for example. In some cases, the delivery device 60 may be a simple sheath. As can be seen, the bi-stable apparatus 110 folds over itself to fit within the delivery device 60. The actuatable device 108 may be advanced distally out of the delivery device 60, such as by being urged distally by a pusher device (not shown) within the delivery device 60. As another example, the actuatable device 108 may be held in position via a pusher device while the delivery device 60 is withdrawn proximally. In either event, FIG. 5 shows a first configuration of the actuatable device 108 as the actuatable device 108 emerges from the delivery device 60. In some cases, the propeller 112 may not emerge from the bi-stable apparatus 110 until the actuatable device 108 reaches its second configuration, as shown in FIG. 6. One of the configurations may be considered as concave and another of the configurations may be considered as convex. It will be appreciated that determination of concave and convex depends upon perspective or point of view.

FIG. 6 shows a first propeller 112 and a second propeller 114. In some cases, the actuatable device 108 only includes a single propeller such as the first propeller 112 or the second propeller 114, that may be located on either side of the bi-stable apparatus 110. In some cases, the actuatable device 108 may include both the first propeller 112 located on a first side of the bi-stable apparatus 110 and the second propeller 114 located on a second side of the bi-stable apparatus 110. In some cases, both the first propeller 112 and the second propeller 114 may have the same pitch. In some cases, the first propeller 112 may have a first pitch and the second propeller 114 may have a second pitch. The first pitch may be equal in magnitude but opposite in direction to the second pitch. The first pitch may differ in both magnitude and direction relative to the second pitch. By having a first pitch that is opposite in direction to that of the second pitch, any blood flow may cause the two propellers to rotate in opposite directions. In some cases, both propellers may have the same pitch direction (with equal or unequal magnitudes), which will cause both propellers to rotate in the same direction.

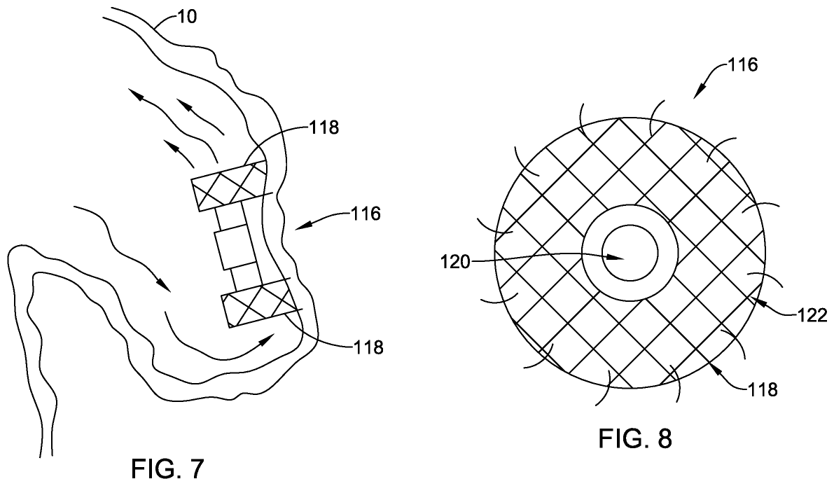

FIG. 7 is a schematic view of an illustrative actuatable device 116 deployed within the LAA 10 and FIG. 8 is an end view of the illustrative actuatable device 116. In some instances, the actuatable device 116 may be an oscillating piston pump. The actuatable device 116 includes anchors 118 that secure the actuatable device 116 in place within the LAA 10. In some cases, the anchors 118 may be braided structures. In some cases, the anchors 118 may be self-expanding anchors, which may be useful when the actuatable device 116 is deployed near a distal end of the LAA 10, and thus the anchors 118 may exert a force on either side of the LAA 10. In some cases, the anchors 118 may include tines that penetrate the wall of the LAA 10. The actuatable device 116 includes a pump intake 120. In some cases, an implantable battery pack, which may or may not be rechargeable via inductive coupling, may be implanted in order to power the actuatable device 116.

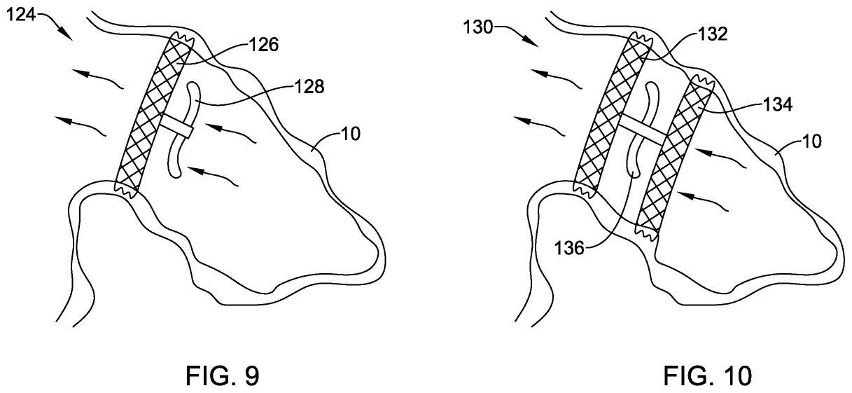

FIG. 9 is a schematic view of an illustrative actuatable device 124 deployed within the LAA 10. The illustrative actuatable device 124 includes a member 126 that spans the LAA 10 and a propeller 128 that is disposed relative to the member 126. In some cases, the member 126 may be a braided or woven ring that spans the LAA 10. Rotation of the propeller 128, which may be caused in part by movement of the member 126 as the heart beats, may help to generate blood flow. It will be appreciated that the actuatable device 124 is adapted to react to blood flow and other movement at all times, not just during times of atrial fibrillation. During times of atrial fibrillation, movement of the propeller 128 may generate sufficient blood flow to create eddies within the LAA 10 is enough to prevent or at least reduce stagnation within the LAA 10. By reducing stagnation, the risk of clot development may be reduced.

FIG. 10 is a schematic view of an illustrative actuatable device 130 deployed within the LAA 10. The illustrative actuatable device 130 is similar in form and function to the actuatable device 124 shown in FIG. 9, but includes a first member 132 and a second member 134 that each span across the LAA 10. The first member 132 and the second member 134 may each be a braided or woven ring that spans the LAA 10, for example. A propeller 136 is disposed between the first member 132 and the second member 134. In some cases, the propeller 136 may be located on a proximal side of the first member 132, or perhaps on a distal side of the second member 134. The actuatable device 130 may include two or more propellers, for example.

Rotation of the propeller 136, which may be caused in part by movement of the first member 132 and/or the second member 134 as the heart beats, may help to generate blood flow. It will be appreciated that the actuatable device 130 is adapted to react to blood flow and other movement at all times, not just during times of atrial fibrillation. During times of atrial fibrillation, movement of the propeller 136 may generate sufficient blood flow to create eddies within the LAA 10 that are enough to prevent or at least reduce stagnation within the LAA 10. By reducing stagnation, the risk of clot development may be reduced.

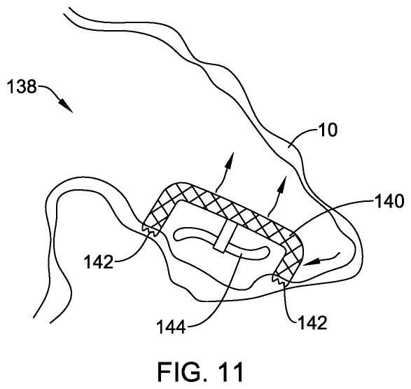

FIG. 11 is a schematic view of an illustrative actuatable device 138 deployed within the LAA 10. The illustrative actuatable device 138 is similar in form and function to the actuatable device 124 shown in FIG. 9, but includes a member 140 that is adapted to be secured along a side wall of the LAA 10, rather than spanning across the LAA 10. The actuatable device 138 includes a member 140 that is secured to the LAA 10 via one or more anchors 142. The anchors 142 may, for example, include tines that extend into the tissue of the LAA 10.

A propeller 144 is disposed relative to the member 140. Rotation of the propeller 144, which may be caused in part by the member 140 flexing as the heart expands and contracts during heart beats, may help to generate blood flow. It will be appreciated that the actuatable device 138 is adapted to react to blood flow and other movement at all times, not just during times of atrial fibrillation. During times of atrial fibrillation, movement of the propeller 144 may generate sufficient blood flow to create eddies within the LAA 10 that are enough to prevent or at least reduce stagnation within the LAA 10. By reducing stagnation, the risk of clot development may be reduced.

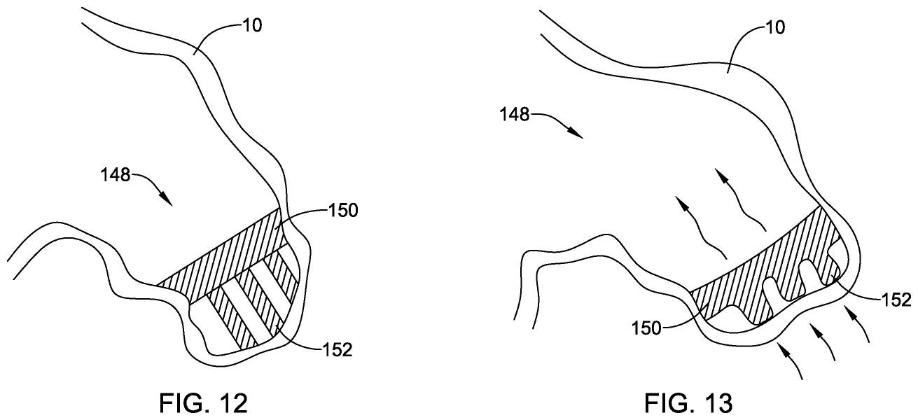

FIG. 12 is a schematic view of an illustrative actuatable device 148 disposed within the LAA 10. The actuatable device 148 includes a first element 150 and a second element 152. In some cases, the first element 150 and the second element 152 may each be formed of elastomeric materials, and thus can elongate and compress in response to movement of the walls of the LAA 10. FIG. 12 shows the second element 152 in a native or relaxed configuration, while FIG. 13 shows the second element 152 in a compressed configuration. The second element 152 will attempt to regain its native or relaxed configuration. As the second element 152 moves, it will create movement within the blood surrounding the second element 152.

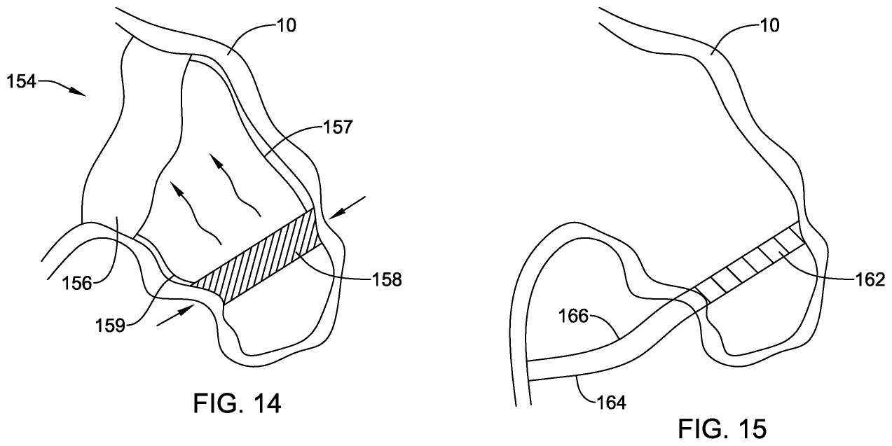

FIG. 14 is a schematic view of an illustrative actuatable device 154 disposed within the LAA 10. The actuatable device 154 fits within the LAA 10, and includes a first element 156 and a second element 158. In some cases, the first element 156 may be a piezoelectric material that generates power in response to movement of the LAA 10. In some cases, the first element 156 captures pectinate muscle motion to power the second element 158. In some cases, the second element 158 may be an EAP (electroactive polymer) that undergoes movement such as contraction or expansion in response to an applied current. In some cases, the second element 158 may be an EAW (electroactive wire) or metallic element. In some cases, the actuatable device 154 may include electrical conductors or signal leads 157 and 159 that extend between the first element 156 and the second element 158 in order to provide power generated by the first element 156 to the second element 158. It will be appreciated that movement of the second element 158 may generate sufficient blood flow to create eddies within the LAA 10 that are enough to prevent or at least reduce stagnation within the LAA 10. By reducing stagnation, the risk of clot development may be reduced.

FIG. 15 is a schematic view of an illustrative actuatable device 160 disposed within the LAA 10. The actuatable device 160 includes an EAP (electroactive polymer) band 162. In some cases, the band 162 may instead be an EAW (electroactive wire) or metallic element. The EAP band 162 may be adapted to undergo movement, such as expansion or contraction, in response to an applied current. In some cases, the EAP band 162 may be powered by signal leads 164 and 166 that are positioned to capture electrical pulses within the cardiac tissue, and to use these captured electrical pulses to power the EAP band 162. It will be appreciated that movement of the EAP band 162 may generate sufficient blood flow to create eddies within the LAA 10 that are enough to prevent or at least reduce stagnation within the LAA 10. By reducing stagnation, the risk of clot development may be reduced.

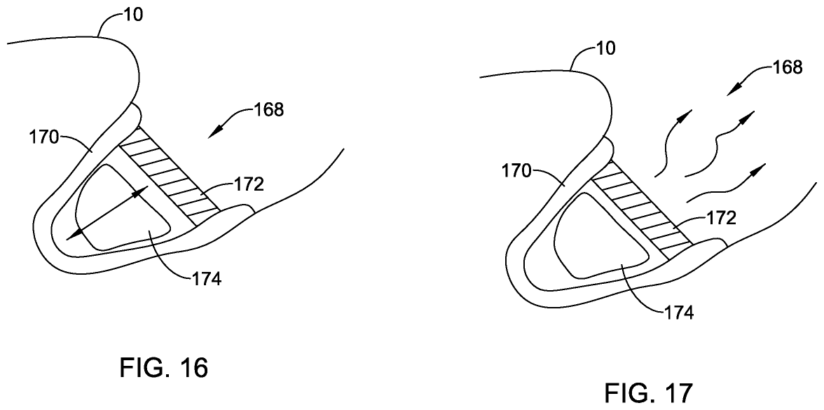

FIGS. 16 through 18 are schematic views of an illustrative actuatable device 168 disposed within the LAA 10. The actuatable device 168 includes a body 170 that is adapted to fit within a distal end of the LAA 10. The actuatable device 168 includes an annular member 172 that spans either side of the body 170. The annular member 172 is adapted to allow blood to flow past the annular member 172. This can include blood flowing distally past the annular member 172, as well as blood flowing proximally past the annular member 172. The actuatable device 168 also includes an agitator 174 that is free to move within the body 170, but is constrained by the annular member 172 from exiting the body 170.

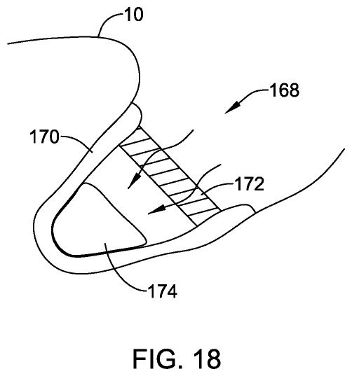

Movement of the heart muscle during various stages of the cardiac cycle may cause the plug member 174 to move back and forth within the body 170. This movement of the agitator 174 may generate sufficient blood flow to create eddies within the LAA 10 that are enough to prevent or at least reduce stagnation within the LAA 10. By reducing stagnation, the risk of clot development may be reduced. The actuatable device 168 may be formed of any of a variety of polymeric materials, including shape memory polymeric materials. FIG. 16 shows the agitator 174 at an intermediate position relative to the annular member 172. FIG. 17 shows the agitator 174 having moved closer to the annular member 172, thereby generating blood flow in a proximal direction. FIG. 18 shows the agitator 174 having moved away from the annular member 172, thereby generating blood flow in a distal direction.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super-elastic nitinol in that the linear elastic and/or non-super-elastic nitinol does not display a substantial “superelastic plateau” or “flag region” in its stress/strain curve like super-elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super-elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super-elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of the devices described herein in order to define a generally smooth outer surface. In other embodiments, however, such a sheath or covering may be absent. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly praraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A method for reducing blood stagnation within a left atrial appendage (LAA) using an actuatable device, the method comprising:
disposing an actuatable device within the LAA that is adapted to create blood flow within the LAA;
actuating the actuatable device in order to reduce blood stagnation within the LAA.

2. The method of claim 16, wherein actuating the actuatable device comprises subjecting the actuatable device to blood flow within the LAA.

3. The method of claim 1, wherein the actuatable device comprises an oscillating piston pump.

4. The method of claim 1, wherein the actuatable device comprises a propeller.

5. The method of claim 4, wherein the actuatable device further comprises a motor adapted to rotate the propeller.

6. The method of claim 4, wherein the propeller is adapted to be actuatable via motion within the LAA.

7. The method of claim 4, wherein the actuatable device comprises a piezoelectric material and a battery.

8. The method of claim 7, wherein normal motion during times without atrial fibrillation is used to charge the battery, and wherein energy is released via the piezoelectric material during times of atrial fibrillation.

9. The method of claim 1, wherein the actuatable device comprises:
a housing adapted to be secured within the LAA;
a housing inlet that is adapted to allow blood to flow into the actuatable device;
a housing outlet that is adapted to allow blood to flow out of the actuatable device;
a power generation element adapted to harness the blood flow to generate power; and
a battery that is charged by the power generated by the power generation element.

10. The method of claim 8, wherein the actuatable device is adapted to recognize periods of atrial fibrillation when the power generated by the power generation element drops below a threshold.

11. A method for reducing blood stagnation within a left atrial appendage (LAA) using an actuatable device, the method comprising:
disposing an actuatable device within the LAA that is adapted to create blood flow within the LAA;
monitoring for indications of atrial fibrillation; and
in response to detecting atrial fibrillation, actuating the actuatable device in order to reduce blood stagnation within the LAA.

12. The method of claim 11, further comprising ceasing operation of the actuatable device when a normal heart rhythm is once again detected.

13. The method of claim 11, wherein atrial fibrillation is detected by recognizing a reduction in generated power.

14. An actuatable device for reducing blood stagnation within a left atrial appendage (LAA), the actuatable device comprising:
structure adapted to anchor the actuatable device within the LAA;
a moveable element that is constrained by the structure but is movable relative to the structure in response to an applied stimuli;
wherein movement of the moveable element in response to the applied stimuli causes blood flow within the LAA, thereby reducing blood stagnation.

15. The actuatable device of claim 14, wherein the applied stimuli comprises movement of cardiac tissue during a cardiac cycle.

16. The actuatable device of claim 14, wherein the applied stimuli comprises an applied current.

17. The actuatable device of claim 14, wherein the moveable element comprises a propeller.

18. The actuatable device of claim 17, further comprising a motor adapted to rotate the propeller.

19. The actuatable device of claim 17, wherein the applied current is externally provided.

20. The actuatable device of claim 17, wherein the applied current is internally generated.
